(19) United States
(12) Patent Application Publication
(10) Pub. No.: US 2012/0009235 A1
(43) Pub. Date: Jan. 12, 2012

(54) STABILIZED PEELING CAPSULE

(75) Inventors: Janina Thilker, Hamburg (DE); Nils Hoffmann, Hamburg (DE)

(73) Assignee: BEIERSDORF AG, Hamburg (DE)

(21) Appl. No.: 13/202,107

(22) PCT Filed: Feb. 16, 2010
(86) PCT No.: PCT/EP10/00950

§ 371 Date: Sep. 26, 2011

(30) Foreign Application Priority Data
Feb. 24, 2009 (DE) 102009010091.1

(51) Int. Cl. A61K 8/11; A61Q 19/00; A61K 31/75

(52) U.S. Cl. 424/401; 424/78.03; 51/295

(57) ABSTRACT

The invention comprises a topically applicable capsule that can be spread by rubbing on the skin, comprising an emulsion-based casing material and a filler material containing a preparation having an abrasive action surrounded by a coating that is applied by a spray technique or dipping bath.

The invention comprises a topically applicable capsule comprising a casing material and a filler material containing a preparation having an abrasive effect, wherein the casing is provided with a coating by means of spray technology or a dipping bath.

WO 2008034549 describes topically applicable capsules that can be rubbed on the skin, comprising an emulsion based casing material and filler material containing a preparation having an abrasive effect, comprising one or more peeling agents having an abrasive effect. The capsule-form preparations are dimensionally stable as coated tablets, capsules, spheres or hollow spheres during storage and removal.

WO 2008034565 describes cosmetic and dermatological peeling preparations in the form of capsules comprising a dimensionally stable capsule casing and a filling.

An additional coating is not disclosed in either document.

DE 102007019373 A1 discloses so-called microcapsules.

The microcapsules are available according to methods known in the prior art. All of the surfactant-stable microcapsules offered on the market can be used as microcapsules, for example, the commercial products (the casing material is given in parentheses in each case) Hallcrest Microcapsules (gelatin, gum arabic), Coletica Thalaspheres (maritime collagen), Lipotec Millicapseln (alginic acid, agar-agar), Induchem Unispheres (lactose, microcrystalline cellulose, hydroxypropyl methylcellulose); Unicerin C30 (lactose, microcrystalline cellulose, hydroxypropyl methylcellulose), Kobo Glycospheres (modified starch, fatty acid esters, phospholipids), Softspheres (modified agar agar) and Kuhs Probiol Nanospheres (phospholipids).

In DE 1027847, DE 2305280, DE 2406021 and DE 1924647, known orally applicable drug forms with film coatings, water-soluble hard-shell capsules, hard gelatin capsules and gelatin capsules resistant to gastric juices are disclosed.

However, none of the set forth prior art documents could show the way to the present invention.

To improve storage stability and breaking resistance of peeling capsules, polymers can be applied to the casing of the capsules. Suitable polymers are cellulose ethers, polyvinyl pyrrolidone, polyacrylates or polymethacrylates as well as Eudragit as described in US 2006127427.

US 2006127427 describes topically applicable capsules that can be rubbed on the skin comprising a casing material and a filler material containing a preparation having an abrasive action comprising one or more water-soluble peeling particles, such as salt or sugar. Polymer-containing, initially water-insoluble, then however water-soluble substances, such as in particular Eudragit® (acrylate copolymer) are described as coating of the capsule. The coating material Eudragit® dissolves in a pH dependent manner and then releases the core of the capsules.

The capsules can be coated as known from the prior art with polishing waxes or hard waxes. These include above all shellac wax, carnauba wax or beeswax as well as further lacquer waxes.

Coating means the casing of a core with sugar layers, for example. Today other coating materials are used with increasing frequency. The applied layer is primarily composed of sugar (classic sugar-coated tablets) or of another film-forming agent (film tablets). The layer is usually colored and can optionally also contain other substances in order to change the properties of the finished drug form, such as the odor or the taste.

Sugar coating is the classic coating method, i.e., the coating of the cores with sugar solutions. High production costs, the difficulties of automating the process and the long duration of production of up to a week per batch cause this method to be replaced more and more by the film tablet.

In cold coating, the sugar solution is applied at normal room temperature, in warm coating (hot coating) a heated sugar syrup is used (approx. 50-60° C.).

The process is carried out in coating pans, in which the cores, the capsules are caused to roll by rotation of the drum. The coating liquid is added and gradually coats the cores. At the same time, a careful drying is carried out, for example, with hot air or UV radiator. In the case of heat-sensitive substances, the drying can also be carried out via the addition of cold air.

The process is repeated until a sufficiently thick and stable layer has formed around the core. This can take up to 50 coating processes. The cores thereby undergo an increase in size and volume.

Quick coating corresponds to the coating described above in its essential operations. Time is often saved by accepting a 70% to 90% lower layer thickness. Furthermore, coating emulsions are used for quick coating. Coated tablets can thus be produced in a few hours.

The disadvantage is that the capsules coated in coating pans have to have an adequate inherent stability for the application in the coating technology and have to be solid enough to resist external mechanical impact. Otherwise, deformation and clogging of the capsules could occur during the coating operation in the pan. Since the sugar solutions applied are aqueous solutions, which dry on the capsule surface, the capsule casing after coating must not release water or at best must be anhydrous, since the coating is otherwise easily detached again and does not firmly surround the capsule. The capsule surface would then become moist and sticky from the dissolved sugar.

In addition to anhydrous wax capsule casings it is also desirable to coat aqueous emulsion casings or to stabilize them through a coating.

The solution according to the invention is a topically applicable capsule that can be rubbed in comprising an aqueous casing material and a filler material surrounded by the casing material, the filler material comprising a preparation having an abrasive action, comprising one or more peeling agents having an abrasive action. Compared to the cosmetic capsules of the prior art, however, the capsule according to the invention is provided with a coating that is applied to the capsule by means of a dipping bath and/or by means of a spray process.

The casing material is preferably formed from an emulsion, comprising one or more waxes, which are solid above 25° C.

The topically applicable capsule is at room temperature a solid, semisolid or dimensionally stable capsule, which thus can be portioned and handled individually.

Many terms such as “balls,” “capsules,” “capsule-shaped preparation” or “beads” can be used in principle to describe the capsules according to the invention, even if different meanings are sometimes assigned to these terms. In particular the meaning of the term “capsule” is not limited here to the precisely defined forms, production methods, ingredients and application possibilities of the pharmaceutical preparations that likewise are referred to as “capsules,” but includes them. In general, according to the invention a capsule is an approximately round or ellipsoid object, clearly distinguishable from its environment, for example, which with light pressure and for example when being grasped for removal from a packaging, changes its shape only insignificantly.

The capsule-shaped preparations according to the invention can have any form, but they are preferably spherical with a volume of 0.1 to 20 ml.

The capsules according to the invention have a size, i.e., average diameter, of from 3, preferably 5, up to 40 mm. The capsules can thus be handled and used individually.

The capsule-shaped preparations according to the invention are dimensionally stable in storage and removal as coated tablets, capsules, spheres, beads or hollow spheres.

Preferably, the capsules described in WO 200803465 and WO 2008034549 are to be used as starting preparations. The disclosures of WO 2008034549 and WO 2008034565 are in their entireties also a disclosure of the present invention.

The casing material of the capsules is advantageously not composed of gelatin or pure wax, but is emulsion-based.

Since aqueous capsule casings are not as stable as, for example, a purely wax casing, and thus more susceptible to mechanical influences, the capsules according to the invention are coated with the aid of a spray process or by means of a dipping bath. Since this type of coating does not take place in the coating pan and the capsules hereby lie on a substrate separated from one another or are drawn through an ethanolic bath, the clogging and breaking of the individual capsules during the coating process can be avoided.

Compared to the prior art, the peeling capsules according to the invention are therefore not to be coated according to known methods, as in coating pans, but instead they are provided with a coating in dipping baths or by means of spray processes.

With the application of the coating with the aid of a spray process the capsules after production are placed and/or transported on a level surface, such as a conveyor belt and sprayed from above with an ethanolic solution. The fine spraying can be carried out with compressed air or as an aerosol with propellant gas, as in the case of industrial spray coatings that are used to coat surfaces. The applied solution is then dried by an air flow, for example. The ethanol evaporates thereby and a thin stabilizing film remains on the capsule. The capsules are turned and sprayed again from the other sides and dried by means of mechanical movement or an air flow, for example.

With the production of a coating by a dipping bath, after production the capsules go through a dipping bath of, for example, ethanolic film former solution or an ethanolic lacquer solution. After the dipping bath the stabilizing coating is likewise advantageously dried by a light air flow.

During the drying or evaporation of the solvent it is also advantageous to turn the capsules slightly so that the coating solution dries uniformly on all parts of the capsule.

The coating according to the invention is advantageously an ethanolic film former solution comprising octylacrylamide/acrylates/butylaminoethyl methacrylate copolymers or acrylates/octylacrylamide copolymer as film forming agent.

Other film forming polymers can also be used advantageously according to the invention like shellac (shellac cera), carnauba or beeswax.

According to the invention, the use of sugar in the casing as well as in the coating is dispensed with.

The coating is advantageously water-insoluble, which is a decisive difference compared to the numerous documents from the prior art.

Advantageously and for improved adhesion of the applied coating on the emulsion casing of the peeling capsule according to the invention, this is first roughened before the spray or dipping treatment. To this end, according to the invention a thin powdery layer is applied to the casing. The powdery layer can be composed of powdery waxes, disaccharides, polysaccharides, cellulose or gum arabic and/or pearlescent pigments.

The powder application is carried out after the formation of the capsules and before the application of the coating described above.

For the powder application, the capsules, for example, lying on an even substrate or the conveyor belt, are atomized or sprinkled with powder from above. This sprinkling operation can be carried out in a comparable manner to the method in which pralines are coated with chocolate. The pralines, in this case the peeling capsules, are thereby located on a conveyor belt and at one point chocolate is poured from above onto the pralines, in this case powder onto the peeling capsule. The coating dries on the transport belt/track (also in the cooling tunnel). The capsules should thereby preferably simultaneously be turned or moved for a uniform application of the powder, for example, by an air flow from below.

The powder application or impingement can also be carried out in a type of coating pan, in which, however, due to the easy deformability of the capsules, only a few or only one capsule should be roughened at the same time. The capsules are hereby turned and at the same time rolled in powder.

For an optically more attractive appearance, pearlescence, color pigments or other colored powder particles can be added to the powdery layer, which color the surface of the capsules. The powder layer is subsequently fixed by the described coating.

The applied powder thickness is advantageously no more than 0.5 mm thick. The powder coating is used primarily to slightly roughen or color the surface of the capsules according to the invention.

Advantageously, pearlescent pigments, such as for example silica+mica+CI 77891 are used as powder layer.

The coating according to the invention has the advantage over the known coatings that this coating can also be used in the case of aqueous emulsion-based capsules. Since a coating pan is not used hereby, the method can also be used for soft capsules.

Moreover, it is possible with the aid of this method of powder application to achieve an optically attractive pearlescence/shimmer effect on the capsules.

Through the treatment with a polymer solution or a wax, the capsule is more dimensionally stable and due to the dried coating an additional peeling effect of the capsules can be achieved.

In the case of the capsules of the prior art, as described, for example in US 2006127427, water-soluble peeling particles such as salt or sugar are enveloped with a coating that is not water-soluble and has to be activated for breakage (by changing the pH value or by activation with water). The coating thus at the same time represents the actual casing and protects the peeling particles.

In contrast, according to the invention an aqueous casing, primarily emulsion-based, is protected with an additional film coating by means of dipping bath or spray process.

According to the invention, the focus is on the protection of the casing to stabilize the peeling beads or to protect the casing and the pearlescent pigments present therein.

According to the invention, a portionable presentation of a face peeling is offered by virtue of the capsule form. The conditioning and cleansing product properties are provided combined in one product.

The capsule according to the invention is thus to be used for skin peeling as the same time as skin conditioning.

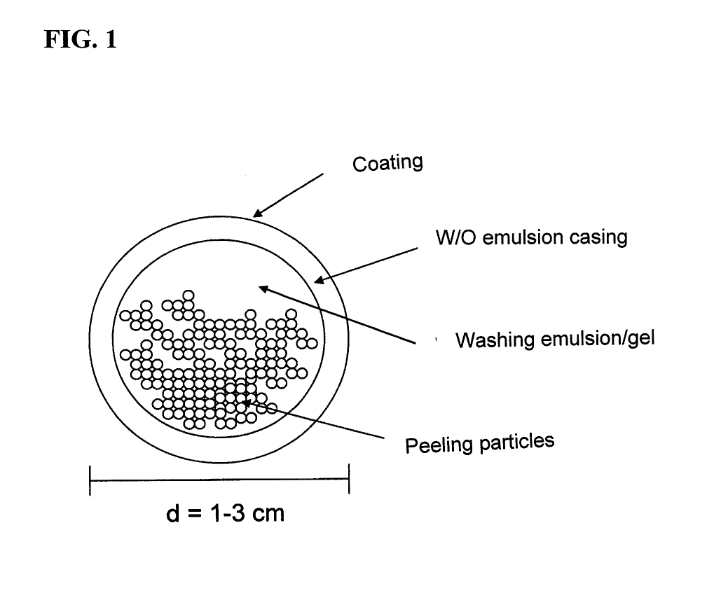

FIG. 1 shows an example of the capsule according to the invention.

The capsule is preferably composed of a
W/O emulsion casing and a
Wax emulsion filling with abrasive particles.

The proportion of the casing is advantageously in the range of 35% by weight to 65% by weight, preferably in the range of 55% by weight, based on the total mass of the capsule.

The proportion of the filling is advantageously in the range of 35% by weight to 65% by weight, in particular in the range of 45% by weight, based on the total mass of the capsule.

The casing is advantageously based on a W/O emulsion.

The peeling agents are advantageously selected from the group of polyethylene, common salt, sea salt, sodium carbonate, sodium bicarbonate, magnesium sulfate, magnesium chloride, sugar, alum clay, sand, plastic particles, pulverized or ground kernels of walnut shells, apricot kernels, peach kernels and/or almond kernels.

### EXAMPLES

The numbers refer to percentage by weight in each case based on the total mass of the casing or the filling.

### Casing Composition:

| 1 | m [%] |
|---|---|
| Cetyl palmitate | 4.0000 |
| Glycerin | 5.0000 |
| Hexamidine diisethionate | 0.0800 |
| Isopropyl palmitate | 25.3200 |
| Octyldodecanol | 29.0000 |
| PEG-45/dodecyl glycol copolymer | 1.3000 |
| Polyethylene | 13.0000 |
| Polyglyceryl-3 diisostearate | 1.3000 |
| Tocopheryl acetate | 1.0000 |
| Water | 20.0000 |

### 2. Constituents of a Hand Peeling Bead Casing

| | m [%] |
|---|---|
| Beeswax | 4.0000 |
| C20-40 Alkyl stearate | 7.5000 |
| Caprylic/capric triglyceride | 12.0000 |
| Cetyl palmitate | 12.0000 |
| Helianthus Annuus (sunflower) seed wax | 4.0000 |
| Hydrogenated coco-glycerides | 45.0000 |
| Isopropyl stearate | 12.0000 |
| Lanolin alcohol (Eucerit ® | 0.5000 |
| Silica dimethyl silylate | 3.0000 |
| Water | ad |

### Filling Composition:

| 3. | m [%] |
|---|---|
| Acrylates/C10-30 alkyl acrylate crosspolymer | 0.6000 |
| BHT | 0.0500 |
| Fragrance | 1.0000 |
| Glycine soya (soybean) oil + | 9.0500 |
| Ricinus Communis (castor) seed oil + propyl gallate | |
| Hydrolyzed silk + phenoxyethanol + | 0.1000 |
| methylparaben + ethylparaben + butylparaben + | |
| isobutylparaben + propylparaben | |
| Lactose + cellulose + ultramarines + | 0.3000 |
| hydroxypropyl methylcellulose + tocopheryl acetate | |
| Lauryl glucoside | 4.4700 |
| Methylparaben | 0.3500 |
| Mineral oil | 35.0000 |
| Oenothera biennis (evening primrose) oil | 0.1000 |
| Phenoxyethanol | 0.8000 |
| Polyethylene | 4.3000 |
| Propylparaben | 0.3500 |
| Sodium myreth sulfate | 7.3200 |
| Water | 36.2100 |

4. Washing Emulsion (with Peeling Particles):

| | m [%] |
|---|---|
| Bis-diglyceryl polyacyladipate-2 | 1.5000 |
| Carbomer | 0.5000 |
| Ceteareth-20 | 2.0000 |
| Cetyl alcohol | 2.2000 |
| Cetyl palmitate | 1.0000 |
| Decyl glucoside | 0.2500 |
| Decyl oleate | 1.0000 |
| Fragrance | 0.3000 |
| Glycerin | 4.3500 |
| Glyceryl stearate | 1.5000 |
| Glycine soja (soybean) oil + | 0.2000 |
| calendula officinalis flower extract | |
| Isopropyl palmitate | 7.5000 |
| Lactose + cellulose + ultramarines + | 0.2500 |
| hydroxypropyl methylcellulose + | |
| tocopheryl acetate | |
| Methylparaben | 0.2000 |
| Methylpropanediol | 2.0000 |
| Mineral oil | 4.5000 |
| PEG-20 glyceryl stearate | 2.5000 |
| Phenoxyethanol | 0.6000 |
| Polyethylene | 4.3000 |
| Prunus Amygdalus Dulcis | 0.2000 |
| (sweet almond) oil | |
| Water | 63.1500 |

5. Washing Gel (with Peeling Particles):

| | m [%] |
|---|---|
| Acrylates/C10-30 alkyl acrylate crosspolymer | 0.6000 |
| BHT | 0.0500 |
| Fragrance | 1.0000 |
| Glycine soja (soybean) oil + | 9.0500 |
| ricinus communis (castor) seed oil + propyl gallate | |
| Hydrolyzed silk + phenoxyethanol + methylparaben + | 0.1000 |
| ethylparaben + butylparaben + isobutylparaben + | |
| propylparaben | |
| Lauryl glucoside | 4.4700 |
| Methylparaben | 0.3500 |
| Mineral oil | 35.0000 |
| Oenothera biennis (evening primrose) oil | 0.1000 |
| Phenoxyethanol | 0.8000 |
| Polyethylene | 4.0000 |
| Polyethylene | 0.3000 |
| Propylparaben | 0.3500 |
| Sodium myreth sulfate | 7.3200 |
| Water | 36.5100 |

### 7. Coating Composition:

| Content | |
|---|---|
| 96% | Alcohol denat. |
| 3% | Octylacrylamide/acrylates/ |
| | butylaminoethyl methacrylate copolymer |
| 1% | Aminomethyl propanol |

The exemplified fillings and casings form the capsules which subsequently are coated according to the invention with the coating by means of a spray process or dipping bath. Advantageously, before the application of the coating, a powder layer is applied in order to improve the roughness and optionally optical acceptance.

1.-8. (canceled)

9. A topically applicable capsule that can be spread on skin by rubbing, wherein the capsule comprises (i) a filler material enclosed by (ii) a casing, the casing being coated by (iii) a coating material, wherein (i) comprises a preparation having an abrasive action, which preparation comprises one or more peeling agents having an abrasive action, (ii) comprises an aqueous casing material, and (iii) has been applied onto the casing by at least one of spraying and dipping, and wherein neither (ii) nor (iii) comprises a sugar.

10. The capsule of claim 9, wherein the casing material comprises an emulsion that comprises one or more waxes having a melting point above 25° C.

11. The capsule of claim 10, wherein the emulsion is a W/O emulsion.

12. The capsule of claim 9, wherein the one or more peeling agents comprise one or more of polyethylene, common salt, sea salt, sodium carbonate, sodium bicarbonate, magnesium sulfate, magnesium chloride, sugar, alum clay, sand, plastic particles, pulverized or ground kernels of walnut shells, pulverized or ground apricot kernels, pulverized or ground peach kernels, and pulverized or ground almond kernels.

13. The capsule of claim 9, wherein (iii) is water-insoluble.

14. The capsule of claim 9, wherein a powder layer is present on the casing.

15. The capsule of claim 14, wherein the powder layer has a thickness of not more than 0.5 mm.

16. The capsule of claim 14, wherein the powder layer comprises at least one of a wax, a disaccharide, a polysaccharide, cellulose, gum arabic, and a pearlescent pigment.

17. The capsule of claim 16, wherein the powder layer comprises a pearlescent pigment.

18. The capsule of claim 9, wherein (iii) comprises at least one of an octylacrylamide/acrylates/butylaminoethyl methacrylate copolymer and an acrylates/-octylacrylamide copolymer.

19. The capsule of claim 9, wherein (ii) accounts for from 35% to 65% by weight of the capsule, based on a total mass of the capsule.

20. The capsule of claim 9, wherein (i) accounts for from 35% to 65% by weight of the capsule, based on a total mass of the capsule.

21. The capsule of claim 9, wherein (ii) accounts for about 45% by weight and (ii) accounts for about 55% by weight of the capsule, based on a total mass of the capsule.

22. The capsule of claim 9, wherein the capsule is spherical.

23. The capsule of claim 9, wherein the capsule has a volume of from 0.1 ml to 20 ml.

24. A plurality of capsules of claim 9, wherein the capsules have an average diameter of from 3 mm to 40 mm.

25. A method of producing a topically applicable capsule that can be spread on skin by rubbing, wherein the method comprises (a) enclosing a filler material by an aqueous casing material, the filler material comprising a preparation with abrasive action that comprises one or more peeling agents with abrasive action, (b) optionally, applying a powder onto the casing material enclosing the filler material, and (c) coating the casing formed in (a) or (b) with a film-forming coating material by at least one of spraying and dipping, neither the casing material nor the coating material comprising a sugar.

26. The method of claim 25, wherein the casing material comprises an emulsion.

27. The method of claim 26, wherein the emulsion is a W/O emulsion comprising one or more waxes which are solid above 25° C.

28. The method of claim 25, wherein the coating material is applied by spraying.
